(19) United States
(12) Patent Application Publication
(10) Pub. No.: US 2022/0020480 A1
(43) Pub. Date: Jan. 20, 2022

(54) SYSTEM AND METHOD FOR MANAGING MEDICAL DEVICES

(71) Applicant: INVACARE CORPORATION, Elyria, OH (US)

(72) Inventors: Matthew E. Monaghan, Chagrin Falls, OH (US); Kevin R. Starkey, Centerville, OH (US)

(21) Appl. No.: 17/376,205

(22) Filed: Jul. 15, 2021

Related U.S. Application Data
(60) Provisional application No. 63/052,647, filed on Jul. 16, 2020.

(51) Int. Cl. G16H 40/40; G16H 10/65; G16H 40/67; G06K 19/077; G06K 7/10; G16H 40/20; B01D 53/04; A61M 16/00; A61M 16/10

(52) U.S. Cl. CPC G16H 40/40; A61M 2205/18; G16H 40/67; G06K 19/07749; G06K 7/10297; G16H 40/20; B01D 53/04; B01D 53/0454; A61M 16/0063; A61M 16/101; G06K 2007/10504; B01D 2253/116; B01D 2256/12; B01D 2257/102; B01D 2259/4533; A61M 2016/1025; A61M 2205/3368; G16H 10/65

(57) ABSTRACT

Systems and methods are provided for managing medical devices. In one embodiment, medical device usage data is stored on the medical device to indicate the usage, health, and alarm or error codes. The usage data is electronically read and assessed against one or more thresholds to determine if the medical device is operating properly and, hence, can be inventoried for reuse, or is need of service or repair. Other embodiments are also disclosed wherein the medical device wirelessly scan its environment to ensure, for example, the device is used with approved accessories or components and personnel. In yet other embodiments, medical devices are provided that can configure themselves for operation by scanning any connected components for component-specific operational data. The operational data is then used to configure the medical device to operate with the component.

This application claims priority to U.S. Prov. Pat. App. Ser. No. 63/052,647 titled “System and Method for Managing Medical Devices” (atty docket no. 12873-07044) and filed on Jul. 16, 2020.

This application incorporates by reference the following patent applications: U.S. Prov. Pat. App. Ser. No. 63/052,694 titled “System and Method for Concentrating Gas” (atty docket no. 12873-07004); U.S. Prov. Pat. App. Ser. No. 63/052,700 titled “System and Method for Concentrating Gas” (atty docket no. 12873-07033); U.S. Prov. Pat. App. Ser. No. 63/052,869 titled “System and Method for Concentrating Gas” (atty docket no. 12873-07041); U.S. Prov. Pat. App. Ser. No. 63/052,533 titled “System and Method for Concentrating Gas” (atty docket no. 12873-07043); and U.S. Prov. Pat. App. Ser. No. 63/052,647 titled “System and Method for Managing Medical Devices” (atty docket no. 12873-07044), all filed on Jul. 16, 2020.

### BACKGROUND

It is not uncommon for medical devices to be provided to patients on either short-term or long-term bases. Examples of such medical devices includes respiratory machines, homecare beds, wheelchairs, etc. One particular type of respiratory machine provided to patients is an oxygen concentrator. Various applications exist for the separation of gaseous mixtures to produce oxygen. For example, the separation of nitrogen from atmospheric air can provide a highly concentrated source of oxygen. These various applications include the provision of elevated concentrations of oxygen for medical patients and flight personnel.

Several existing product gas or oxygen concentrating systems and methods, for example, are disclosed in U.S. Pat. Nos. 4,449,990, 5,906,672, 5,917,135, 5,988,165, 7,294,170, 7,455,717, 7,722,700, 7,875,105, 8,062,003, 8,070,853, 8,668,767, 9,132,377, 9,266,053, and 10,010,696 which are commonly assigned to Invacare Corporation of Elyria, Ohio and fully incorporated herein by reference.

Such systems are known to be either stationary, transportable, or portable. Stationary systems are intended to remain in one location such as, for example, a user's bedroom or living room. Transportable systems are intended to be moved from location to location and often include wheels or other mechanisms to facilitate movement. Portable systems are intended to be carried with the user such as, for example, via a shoulder strap or similar accessory.

In one aspect, these medical devices are inventoried and re-used by medical device providers. The adequacy of the inventory depends on which and how many devices need to be serviced and to what degree. It is desirable to address these and other aspects of managing medical devices.

### SUMMARY

Systems and methods are provided for managing medical devices. In one embodiment, the ability to manage medical devices based on their usage history is provided. In another embodiment, the ability to manage medical devices is provided based on their diagnostic history. In yet another embodiment, the ability to manage medical devices is provided based on determining which devices need to be serviced or repaired before they can be inventoried for re-use. Other embodiments are also disclosed.

### BRIEF DESCRIPTION OF THE DRAWINGS

In the accompanying drawings which are incorporated in and constitute a part of the specification, embodiments of the inventions are illustrated, which, together with a general description of the inventions given above, and the detailed descriptions given below, serve to example the principles of the inventions.

FIG. 1 shows one embodiment of a medical device.

FIG. 2 is one embodiment of a block diagram illustrating various components of a control system of a medical device.

FIG. 3 is one embodiment of a system for managing a medical device.

FIG. 4 illustrates one embodiment of a system and method for managing a medical device inventory.

FIG. 5 illustrates another embodiment of a system and method for managing a medical device inventory.

FIG. 6 illustrates yet another embodiment of a system and method for managing a medical device inventory.

FIG. 7 illustrates an embodiment of a system and method for reading and writing medical device data.

FIG. 8-9 illustrate embodiments of systems and methods for managing components of a medical device.

FIGS. 10A-10C, 11A-11C, and 12 illustrate embodiments of systems and methods for automatically configuring a medical device.

FIG. 13A-C illustrate embodiments of a system and method for power management of RFID data processes and circuits.

FIG. 14 illustrated one embodiment of a data structure for conveying information between components.

### DESCRIPTION

As described herein, when one or more components are described or shown as being connected, joined, affixed, coupled, attached, or otherwise interconnected, such interconnection may be direct as between the components or may be indirect such as through the use of one or more intermediary components. Also, as described herein, reference to a member, component, or portion shall not be limited to a single structural member, component, element, or portion but can include an assembly of components, members, elements, or portions.

Embodiments of the present inventions provide, for example, the ability to assess the condition of a medical device. This includes determining whether the medical device can be inventoried for re-use and/or requires service or should be serviced soon. The ability to assess the condition of an already inventoried medical device is also provided. This allows the device to be checked while already in inventory before being sent to a user or a patient to determine if the device should be serviced prior to being sent. In this manner, medical devices can be efficiently identified for service thereby reducing the need to retrieve such medical devices from users.

Embodiments of the present inventions also provide, for example, a “smart” technology using wireless communication (e.g., RFID) with a medical or other device to enhance product management and exchange of on-board and off-board information throughout the life of the product. The “smart” technology also provides the ability to make handling the unit as an item of inventory which is being stored, dispatched to a user environment, retrieved, diagnosed, serviced, tracked and stored, more efficient for those involved in handling the unit, tracking changes in status of the unit, collecting and trending data on the unit, its performance, and defects, for example.

Embodiments of the “smart” technology also provide logic for using data of the device to help prevent unintended use, use in unsafe conditions, to track the identity and information about who, where and when people are interacting with the device for logging, investigative, forensic or other purposes. In some cases, more than one device, each having its own RFID tag may have interoperable functionality, such as one unit reading for compatibility of another component, accessory and/or system. Further yet, embodiments of the “smart” technology provide logic and the ability to use data from the system to seek reimbursement, confirm use for billing or billing justification, to determine patient compliance with amount and conditions of use and to determine whether use was in compliance with requirements. This data can include, for example, patient name, address, physical state (weight, height, blood type, etc.), insurance information (e.g., provider, policy number, etc.), location (e.g., facility name, floor, room, division, level, etc.). The data can also include, for example, medical device manufacturer, model number, serial number, location, etc. Other examples of RFID data are provided throughout the disclosure.

In one embodiment, the medical device can be an oxygen concentrator which provides high purity oxygen to patients. Oxygen concentrators include many components such as, for example, compressors, valves, sieve beds which are used to separate nitrogen from room air to produce oxygen, motors, filters, etc. Over time these components may need to be serviced due to component wear and/or reduced efficiency based on usage and environment.

For example, compressors and valves use seals to ensure against gas leakage. Compressors and valves also include mechanical components such as, for example, rods, pistons, bearings, heads, actuators, etc. Sieve beds that are used to separate gases employ a granular sieve material that can mechanically break down over time (known as dusting) due to the dynamic pressures of air being cyclically fed into the sieve beds. Sieve beds can also deteriorate based on moisture being present in the air that is fed into the sieve beds. Furthermore, the control systems that operate oxygen concentrators rely on one or more sensors including, for example, pressure, temperature, oxygen, flow, etc. the failure of any one or more these components can result in a medical device that needs service before it can be inventoried for reuse. Furthermore, many of these components may need servicing based on a schedule in order to prevent or minimize the possibility of their failure while away from the medical device provider and with the patient.

Embodiments of the present inventions provide the ability to scan a medical device to determine whether it can be sent to inventory for reuse or should be sent to be serviced. The scanning can be by any appropriate communication means including, for example, Radio-Frequency Identification (RFID) technology, Near-Field Communication (NFC) technology, Bluetooth™ technology, local wireless network such as Wireless Local Area Network (WLAN) technology (Wi-Fi) (or IEEE 822.11), or cellular communication technology. The scanning transmits data from the medical device to allow assessment of the medical device. The data can be any diagnostic and/or usage data associated with the medical device's operation, components, and/or usage.

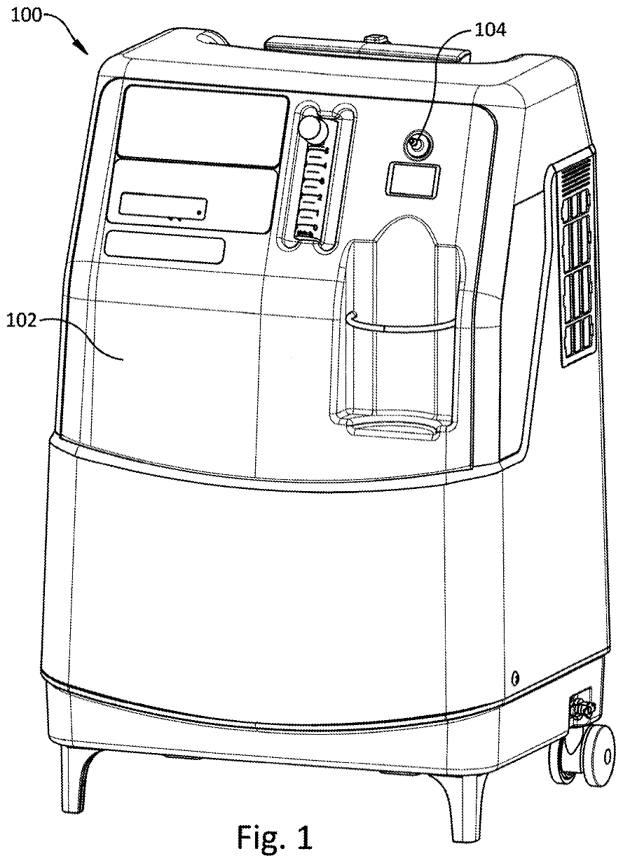

Illustrated in FIG. 1 is one embodiment of an oxygen system 100. The system may be stationary such as, for example, for use in a hospital or a patient's home. The system can also be ambulatory or mobile such as, for example, for use by a patient when they are away from home. The system can be configured in a manner to allow the patient to carry the system such as, for example, through an over the shoulder strap or through an arrangement whereby the system includes a handle and wheels. Other mobility configurations are also included.

Oxygen system 100 includes a housing 102, which can be in one or more sections. Housing 102 includes a plurality of openings for the intake and discharge of various gases such as, for example, the intake of room air and the discharge of nitrogen and other gases. Oxygen system 100 generally intakes room air, which is mostly comprised of oxygen and nitrogen, and separates the nitrogen from the oxygen. The oxygen is stored in one or more internal or external storage or product tanks and the nitrogen is discharged back into the room air. For example, the oxygen gas may be discharged through port 104 to a patient through tubing and nasal cannula. Alternatively, the oxygen gas may be discharged through a supplemental port to an oxygen cylinder filling device, such as HOMEFILL® that is manufactured by Invacare Corp. of Elyria, Ohio, USA.

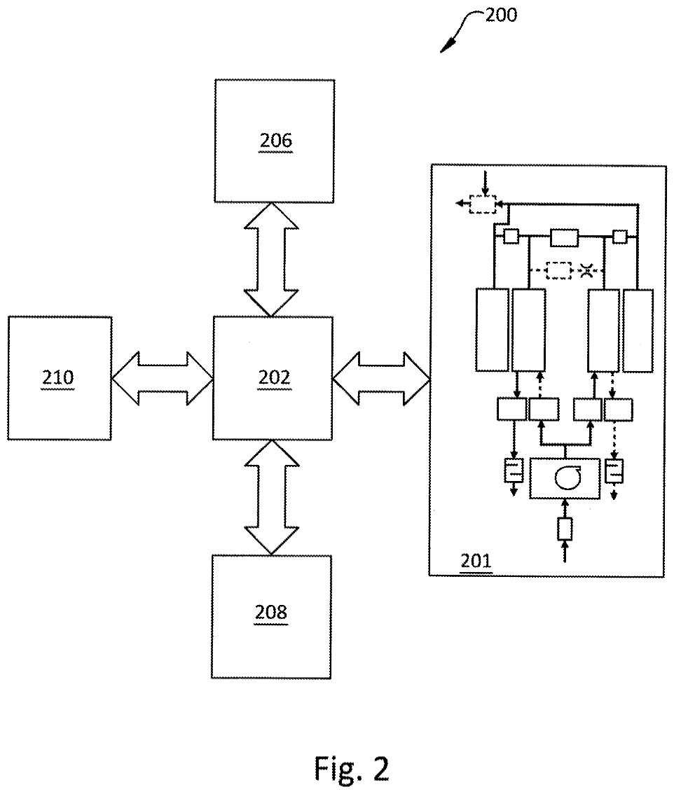

FIG. 2 illustrates one embodiment of a medical device control system 200. System 200 includes the controller 202 for controlling the medical device, which can be an oxygen concentrator system. System 200 further includes inputs and outputs 206, which can include, for example, displays, buttons, speakers, communication ports, etc. System 200 also includes memory 208 associated with controller 202 for containing storage for data, logic, and software instructions. Further yet, system 200 includes a wireless communication device 210, which can be for example, an RFID tag. The RFID tag can be passive, active, and/or semi-passive. In one embodiment, the RFID tag includes a controller and memory into which medical device assessment data can be stored and read from. In other embodiments, the medical device assessment data can be stored in memory 208 and access therefrom.

The medical device assessment data can include, for example, one or more of the following: usage data (including component hours, cycles, runtime, etc.), diagnostic data (including error codes, messages, alarms, etc.), location data (room name/number, building name/number, floor or level, etc.), device data (including serial number or other identification data, etc.), operational data (including oxygen purity, shift or cycle pressures, temperatures, averages thereof, etc.) This description is intended to be exemplary and not limiting. The medical device assessment data can include any data helpful to assess the status of the medical device in the case of, for example, assessing whether the device needs service and/or replacement. This information or data is also helpful during the troubleshooting and repair process because it can either directly and/or indirectly identify system components that need to be replaced or repaired.

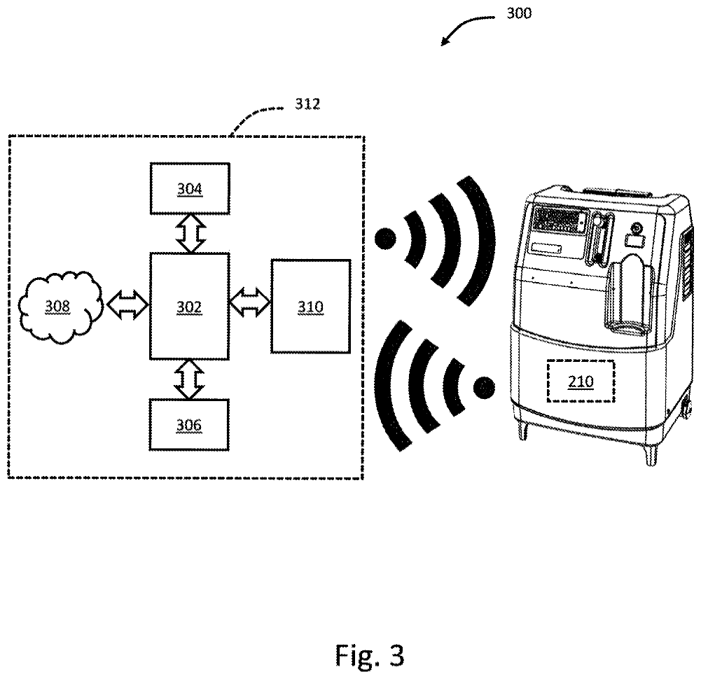

FIG. 3 illustrates a system and method 300 for communicating with the medical device. In one embodiment, the medical device uses RFID technology including, for example, RFID tag 210. In other embodiments, any of the previously described wireless communication technologies can be used. A system 312 for reading (and/or writing to) the RFID tag 210 is provided that includes a controller 302, I/O 304 which can include a touch screen or other user input and output device(s), a memory 306 for storing data and software logic, an optional connection to a network 308, and a scanner or reader 310 for reading device data (and which can also write data in other embodiments). Not all of these components are required for system 312 but are illustrative of one embodiment. System 312 can be a laptop computer, a tablet computer, a smart phone, handheld RFID read/write scanner, a fixed location scanner (e.g., doorway, shelf, etc.) or any of the equivalent.

In operation, system 312 scans the RFID tag 210 associated with the medical device to retrieve the data contained thereon. System 312 generates a radio frequency signal that is received by RFID tag 210. The radio frequency signal can be of any appropriate frequency including, for example, low frequency (LF) (e.g., 125 kHz or 134 kHz), high frequency (HF) (e.g., 13.56 MHz), and/or ultrahigh frequency (UHF) (e.g., 860-960 MHz). Low-frequency RFID provides a range of up to 10 cm. High-frequency RFID provides a range of up to 1 meter. Ultrahigh frequency RFID provides a range of up to 10 to 15 meters. Any one or more of these frequencies can be used.

The RFID signal is received by RFID tag 210 and RFID tag 210 can respond by transmitting and RFID signal containing data within its memory (and/or device controller memory 208). As described above, in one embodiment this data contains medical device assessment data. System 312 can also write data to RFID tag 210 by this same RFID process. The RFID signals create a communication link between system 312 and the memory and controller within the RFID tag 210 (and/or controller 202 and memory 208 in the medical device). In one embodiment, system 312 includes logic (or software instructions) within memory 306 to assess whether the medical device needs to be serviced or can be inventoried for reuse. In other embodiments, system 312 can convey the data to network 308 for assessment of the medical device. In other embodiments, system 312 can receive data from network 308 to be written to or saved in the RFID tag 210 of the medical device. In yet other embodiments, data can be written or saved to RFID tag 210 via user input through an interactive RFID user interface or other means. Hence, the logic for assessing the medical device can reside in any one or more locations.

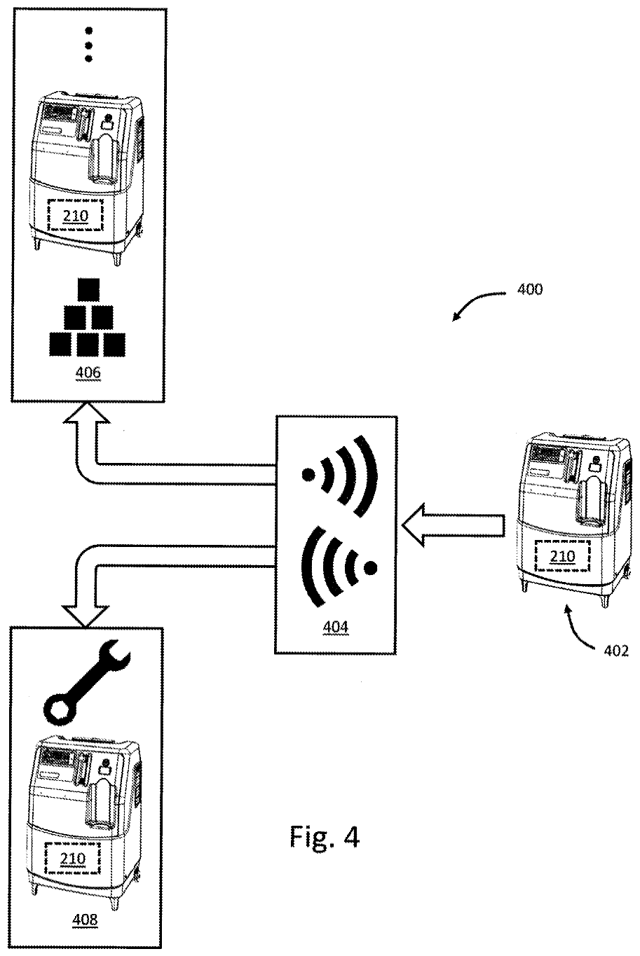

FIG. 4 illustrates one embodiment of a system and method 400 for assessing whether a medical device needs to be serviced or can be inventoried for reuse. Medical device 402 is scanned by assessment system 404 to obtain the medical device's assessment data. Embodiments of assessment systems are shown and described in connection with FIGS. 3-6 and utilize RFID communication technology, though any of the previously described technologies may be employed. In this embodiment, the medical device assessment data can include one or more of compressor usage hours and/or active alarm codes. In other embodiments, any one or more of the previously described data can also be included such as, for example, device health data including average oxygen purity, average cycle or shift pressure, and average operating temperature. If assessment system 404 determines the data does not indicate service is required, the medical device can be designated as suitable for being inventoried for reuse (which may include conventional cleaning or sanitizing of all units that are to be inventoried after usage.)

For example, data that indicates the compressor usage hours are low (e.g., below 4,000 hours (or 6 months), below 26,000 hours (or 3 years), or some other threshold) indicates the device does not need service and can be sent to be inventoried at 406 for reuse. Similarly, data that indicates there are no active alarm codes signifies the device does not need service and can be sent to be inventoried at 406 for reuse. Also, data that indicates the average oxygen purity, shift pressure, and operating temperature are within appropriate ranges can also signify the device does not need service and can be sent to be inventoried at 406 for reuse. For example, if the data indicates the average oxygen purity is above 85%, the device is within the operating range for oxygen purity. Also, for example, if the data indicates the average shift pressure is within 15-31 PSI, the device is within the operating range for shift pressure. Further, if the data indicates the average temperature is below 125 degrees Fahrenheit, the device is within the operating range for temperature. Other values than those described herein can be used as thresholds for proper operating ranges. However, if any one or more of the data are out of or beyond appropriate operating ranges or thresholds, the medical device can be assessed or designated to be sent for service at 408. Service can involve repairing or replacing any one or more of the components that caused the medical device assessment data to indicate service is necessary. In this manner, medical devices such as, for example, oxygen concentrators, can be assessed for inventory management or service when the concentrators are returning from the field (or patient use).

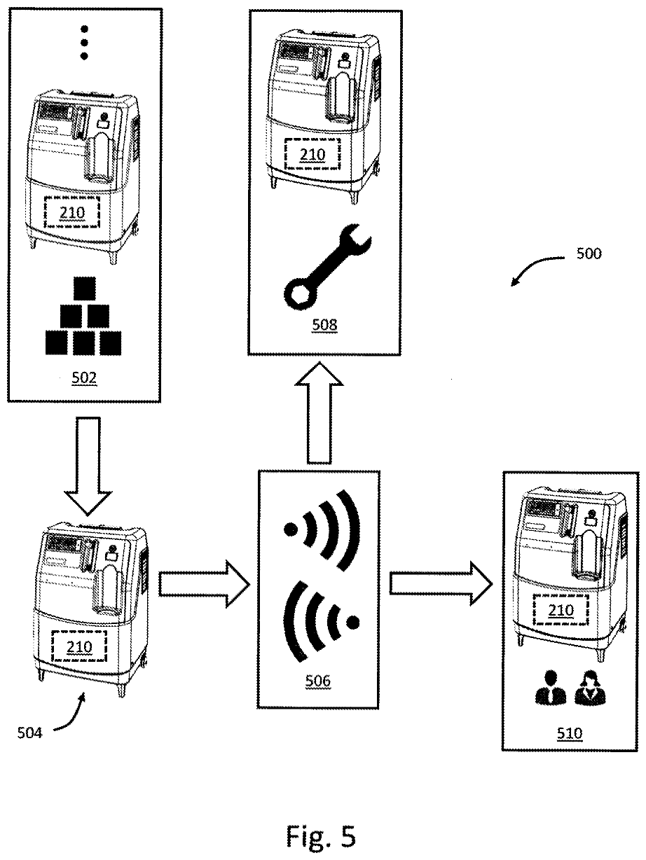

FIG. 5 illustrates one embodiment of a system and method 500 for assessing whether a medical device 504 in inventory at 502 is ready to be for use. Medical device 504, which may reside in inventory, is scanned by assessment system 506 to obtain the medical device's assessment data. As previously described, embodiments of assessment systems are shown and described in connection with FIGS. 3-4 and 6 and utilize RFID communication technology, though any of the previously described technologies may be employed. The assessment data can include any one or more of the previously described data including, for example, compressor usage hours, active alarm codes, and/or device health data including average oxygen purity, average cycle or shift pressure, and average operating temperature.

If assessment system 506 determines the data does not indicate service is required, the medical device can be taken from inventory for use at 510. If the assessment system 506 determines one or more of the data are out of or beyond appropriate operating ranges or thresholds, the device which has been in inventory at 502 can be sent were designated for service at 508. The data assessments described above in connection with FIGS. 3 and 4 are also applicable here in connection with the embodiment of FIG. 5. As such, medical devices that may already be in inventory can be checked or confirmed that they do not need to be serviced when it is time for them to be taken from inventory for use. This provides the ability to capture any medical devices that may have been placed in inventory but require service prior to being put back in use.

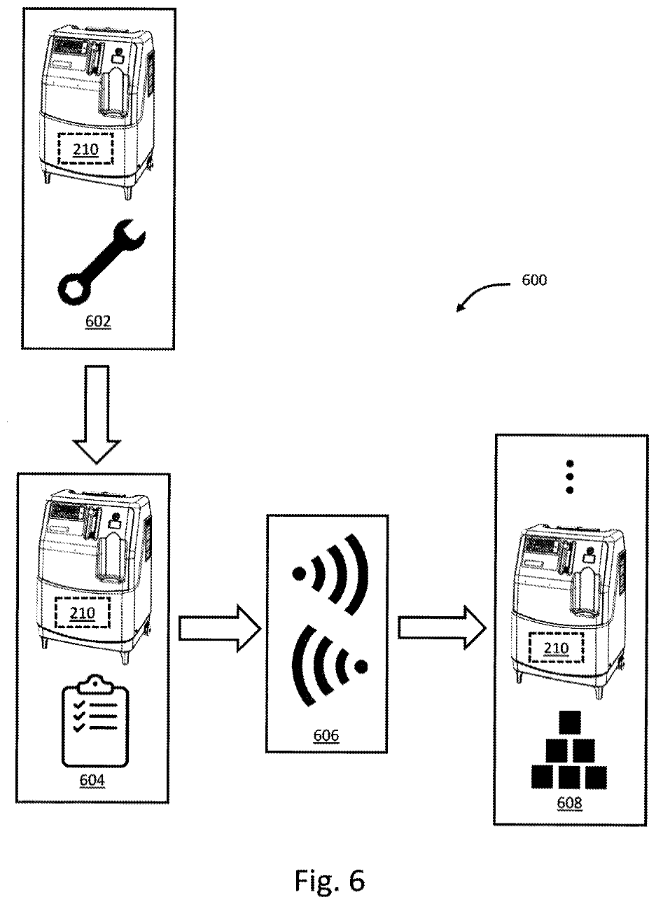

FIG. 6 illustrates one embodiment of a system and method 600 for assessing whether a medical device 604 that has been serviced at 602 is ready to be placed in inventory at 608. Medical device 604, which may have been serviced, is operated for a time period (e.g., 12-24 hours, for example) to allow its control system to collect the assessment data described herein. Thereafter, medical device 604 is scanned by assessment system 606 to obtain the medical device's assessment data. As previously described, embodiments of assessment systems are shown and described in connection with FIGS. 3-5 and utilize RFID communication technology, though any of the previously described technologies may be employed. The assessment data can include any one or more of the previously described data including, for example, compressor usage hours, active and/or previously triggered alarm codes, and/or device health data including the average, minimum, and/or maximum values for: oxygen purity, cycle or shift pressure, and operating temperature. This assessment data is meant to be illustrative and any other data representative of the health or status of the medical device can be used.

If assessment system 606 determines the data does not indicate further service is required, the medical device 604 is ready to be placed in inventory at 608. If the assessment system 606 determines one or more of the data are out of or beyond appropriate operating ranges or thresholds, the device which has been serviced can be returned to service at 602. If the data indicates medical device 604 is operating correctly, medical device 604 can be placed in inventory at 608. Data indicative of the results of these assessments (e.g., inventory or service required) can be stored in an equipment provider database along with date, place, and device usage or health data that was used in making the assessment. The data assessments described above in connection with FIGS. 3-5 are also applicable here in connection with the embodiment of FIG. 6. In this way, medical devices that may have been serviced can be checked or confirmed that they do not need to be further serviced and can be confidently placed in inventory for use.

Further, in any of the aforementioned embodiments, the assessment systems may also track medical devices entering (e.g., FIG. 4), exiting (e.g., FIG. 5), or moving within the facility (e.g., FIG. 6). As shown in these embodiments, the assessment systems can determine whether each medical device is located in, for example, service, inventory, or has exited the facility. Moreover, in the case of service and inventory, additional RFID scanners can be used to track the location of the medical devices within each of these spaces or functions. For example, if the medical device is in service, the additional RFID scanners can be located to indicate whether the medical device is in compressor repair, sieve bed repair, valve repair, post-repair testing, etc. If the medical device is in inventory, the additional RFID scanners can be located to indicate which portion of inventory (e.g., shelf location, area, building, etc.) the medical device is located. In these examples the device's serial number can be read by each of the RFID scanners to associate or track a medical device within areas of the facility.

The systems and methods described herein can be embodied in computer-implemented technology. This includes hardware or software logic for causing controllers and/or microprocessors to execute instructions for accomplishing the functions and steps described herein. For example, the logic described herein for assessing the medical device data obtained via the RFID technology (or other wireless technology) can be embodied in hardware and/or software (including computer-readable mediums). Also described herein, the systems and methods may be implemented using network technology involving server and client type architecture. Further yet, database technology can be used for managing the inventory and that database technology may employ local and/or remote databases.

Thus, the embodiments of systems and methods described herein provide for inventory management of medical devices. This includes the ability to track medical devices with RFID technology within facilities (including inventory and service locations). This also includes the ability to use RFID technology to quickly scan inventory shelves to assess the inventory that is physically present. This further includes the ability to use RFID technology to select inventory units for use based on usage data (e.g., low compressor hours) and/or device health data (e.g., the absence of active alarm codes).

This also includes the ability to simplify the troubleshooting process. All the returning medical devices can be quickly scanned by RFID technology to record their assessment data including usage data (e.g., compressor hours) device serial number, and/or device health data (e.g., active alarm or error codes, average oxygen purity, average shift pressure, average operating temperature, etc.) Still further, after service or repair and test operation (e.g., overnight) RFID technology (or similar technology including, for example, Near Field Communication (NFC), Bluetooth, Wi-Fi, etc.) can be used to scan the medical device to identify devices that have failed, need further service, or are ready to be placed in inventory by operating as expected.

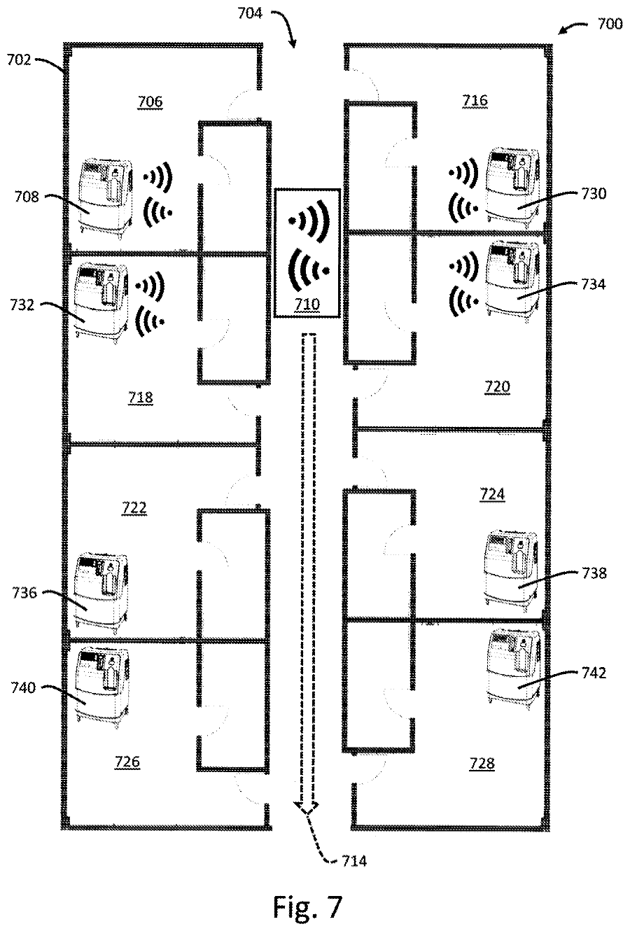

Referring now to FIG. 7, one embodiment of a system and method for communicating with one or more medical devices is shown. The medical device can be any medical device having a communication system or means as described herein including, for example, RFID. In one embodiment, the medical device can be a respiratory device such as an oxygen concentrator (embodiments of which are/have been described within the present disclosure), ventilator or CPAP device. In other embodiments, the medical device can be an intravenous machine, dialysis machine, etc.

The embodiment of FIG. 7 allows one or more medical devices 708, 730-742, for example, to be polled or communicated with to obtain their data from a location within or outside of the room in which the medical devices are located. The embodiment of FIG. 7 also allows such communication to occur through RFID, which provides connectivity without the cost or complications of other communication networks such as Wi-Fi, etc., though such networks may also be used in alternative embodiments herein.

The embodiment of FIG. 7 will now be described in the context of a medical facility 702 such as, for example, a hospital, nursing home, short or long-term care facility, etc. Facility 702 typically includes one or more rooms such as, for example, rooms 706 and 716-728 and hallways 704. One or more of the rooms can contain at least one medical device (e.g., 708, 730-742) such as, for example, an oxygen concentrator. Each medical device can include, for example, an RFID tag in logic for reading and writing data to the RFID tag as described herein. This includes, for example, medical device usage data, health data, location data, patient/user data, etc. As each medical device 708, 730-742 operates, its control system writes or stores the appropriate data to the RFID tag.

The embodiment of FIG. 7 further includes a system 710 for reading and/or writing to the RFID tags of medical devices 708, 730-742. In one embodiment, system 710 is similar to system 312 previously described in connection with FIG. 3. System 710 can be in any physical form including, for example, a handheld scanner unit, tablet, laptop, personal computer, etc. Scanner 710 can communicate with medical devices 708, 730-742 from outside of rooms 706 and 716-728. This provides efficiency as scanner 710 does not have to enter each room in order to communicate with a medical device. This also maintains room isolation and quarantine where necessary, room privacy, minimizes room interruptions, etc. In other embodiments, scanner 710 can also communicate with the medical devices from within the rooms.

Scanner 710 can operate within a hallway 704 or other accessway and poll or communicate with the medical devices 708, 730-742. Each medical device is scanned by scanner 710 and provides its data in response. In one embodiment, scanner 710 creates an RFID connection with each medical device within the range of scanner 710. As previously described, the data of each medical device can include a unique device identifier (e.g., serial number, etc.) along with other device data. Medical devices 708, 730-742 collectively provide an off-line repository of updated medical and medical device data that can be read at any desired interval by scanner 710. This repository of data is maintained by each medical device as it operates and stores its data within the RFID tag for communication with scanner 710. In response to being scanned, each medical device provides its data to scanner 710. Scanner 710 can store the medical data, upload the data to a cloud-based server or database, and or perform other operations such as analytics, reports, and writing data back to the medical device (e.g., RFID tag).

Scanner 710 can move along hallway 704 as shown by arrow 714 to communicate with one or more medical devices in the facility 702 or the particular floor or level of the facility 702. In yet other embodiments, scanner 710 can be a directional scanner allowing for not only RFID data communication but also determination of physical location of specific units. For example, a directional scanner can be pointed in the general direction of scanning to determine if a medical device is present in that direction or general location. In this manner, a determination from the medical device health data of which medical devices need service or maintenance due to component wear/usage and/or alarm or service codes/errors being present in the medical device health data can be obtained quietly and privately without entering, for example, a patient, hospital or other facility room. If any one or more of such codes or data is present, the medical device can be retrieved from its location and be sent for servicing/repair. A replacement device can then be put in service for the unit taken away.

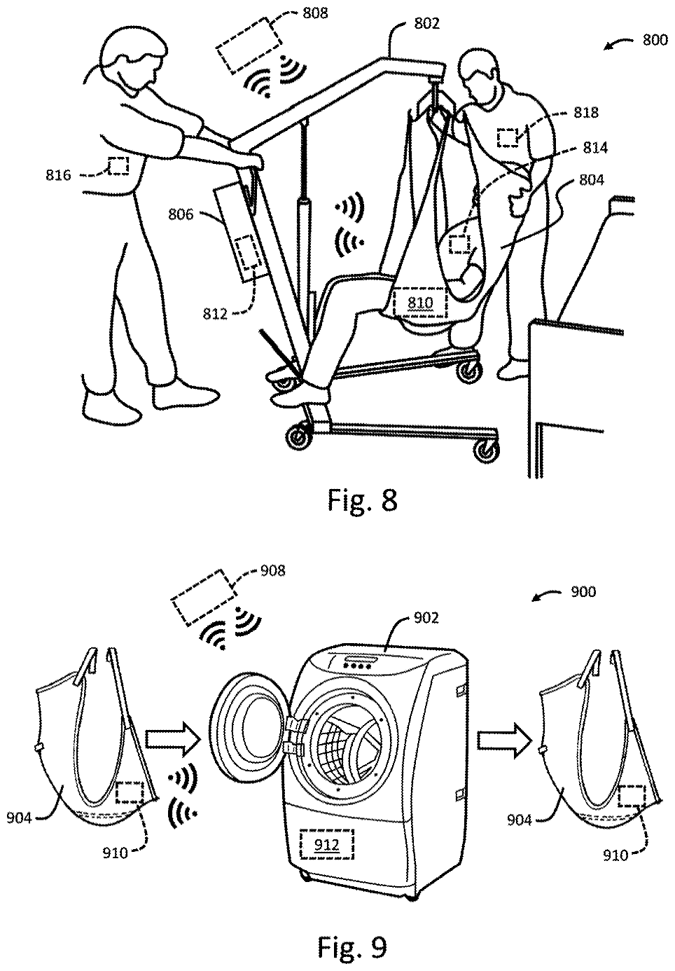

Referring now to FIGS. 8 and 9, another embodiment 800 of a system and method for communicating with a medical device is provided. In this embodiment, the medical device can be a patient lift 802 having a patient sling 804. Patient lifts are used for moving patients from one location to another such as for example, from a bed to another bed or wheelchair, etc. Examples of patient lifts are described in U.S. Pat. Nos. 8,272,084, 8,250,687, and PCT/IB2018/059565 (published as WO 2019/124059), assigned to Invacare and Invacare International GMBH, which are hereby incorporated by reference. Patient lifts use patient slings 804 to support a patient during movement. Slings 804 can be in various sizes and configurations and one embodiment is shown in FIGS. 8 and 9. Slings 804/904 are made of strong durable material such as polyester, nylon, Kevlar, etc. and are capable of supporting various weights including up to, for example, 600 pounds or more. Each sling typically includes one or more straps/handles for connecting the sling to the patient lift.

In the embodiment of FIG. 8, sling 804 can communicate with patient lift 806 and or a scanner 808 and transmit and/or receive data. Patient lift 806 can includes its own scanner 812 and/or its own RFID tag (within a control box 806) and can also transmit and/or receive data to/from scanner 808. Scanner 808 can be in any physical form including, for example, a dedicated room scanner, a handheld scanner unit, tablet, laptop, personal computer, etc., In one embodiment, patient lift 802 reads and/or writes data to sling RFID tag 810. This includes reading and updating sling usage information including one or more of the number of times sling 804 has been used, how long sling 804 has been in use since beginning of service, how many wash cycles sling 804 has experienced, serial or identification number, manufacturer, specification (e.g., weight capacity, type), authentication data to ensure the sling is being used with an authorized patient lift or vice-versa, sling health data including when sling 804 was made, expected life of sling 804 or replacement date, etc.

Patient lift 802 can poll or scan sling RFID tag 810 to obtain the aforementioned data to determine if sling 804 is safe for use including determining whether sling 804 is authorized for use with the patient lift, whether sling 804 is past its service life and needs replacing as determined by any one or more the usage data, wash cycle data, replacement data, etc., being in excess of predetermined threshold levels either contained on the RFID tag or with the scanner's logic. This same data (and additional data) can also be maintained by the patient lift to form its own data or health data set that can be read and written to by, for example, scanner 808. Hence, either or both patient lift 802 and sling 804 can have medical device data (e.g., usage, health, identification, alarm, etc.) associated therewith that can be polled or scanned using RFID or other communication technology to determine proper operation of the device (including non-operation, service, repair, and/or replacement). This reduces injury and unsafe conditions for patients and aides by providing notice through device data that patient lift 802 and/or sling 804 should not be used or should be replaced soon.

In this regard, patient lift 802 and/or scanner 808 can include one or more notifications or displays indicating sling 804 should not be used. These can be activated if the medical device data exceeds one or more of the previous mentioned thresholds. In yet another embodiment, the patient and caregivers can include an RFID tag for identification purposes. For example, the patient's RFID tag 814 can include data indicating their physical state such as height and/or weight, name, room number, associated medical staff, etc. The caregiver's RFID tag 816 can include identification data and/or data indicating they are trained, certified, or authorized for patient lifting and transporting. In operation, patient lift 802 and/or scanner 808 can scan the RFID tag data of the patient (814) and caregivers (816) in order to determine if the lift 802 and sling 804 are rated for the particular patient (e.g., the patient weight is below the maximum lift and sling weight rating) and that the appropriate number of caregivers (e.g., two) are present. If these thresholds are satisfied, patient lift 802 can authorize or be authorized for use. If not, patient lift 802 can be disabled to prevent the occurrence of an unsafe condition and an alarm, display and/or notification can be generated.

The alarms, notifications, and/or displays can take the form of one or more visual and/or auditory signals generated from or by lift control box 806 and/or scanner 808. The visual signals can include colored displays (e.g., yellow or red displays or lights (flashing or otherwise) indicating sling 804 requires attention, needs to be replaced soon, or requires replacement). The auditory signals can include, for example, one or more beeps, buzzes, voice notifications and/or alarms indicating the same. Other forms of notifications/displays can also be used. Still further, such notifications can also be stored as data on the RFID tag of the device and/or transmitted to a remote server for device management (e.g., service, repair, inventory, re-ordering, etc.)

FIG. 9 illustrates another embodiment 900 for a system and method for managing a medical device. In this embodiment, the medical device is a patient sling 904, which requires cleaning or washing periodically. Cleaning and washing cycles can be used as a form of usage or component wear data for patient slings and other similar medical devices. The number of cleaning and washing cycles can contribute to the wear of the medical device and hence the duration of the device's service life. In this embodiment, patient sling 904 includes an RFID tag 910 for communicating with a washing machine 902 that has a scanner 912 associated therewith. Additionally, or alternatively, sling RFID tag 910 can communicate with scanner 908. Scanner 908 can be in any physical form including, for example, a room mounted scanner, a handheld scanner unit, tablet, laptop, personal computer, etc.

The sling RFID tag 910 is scanned by scanner 912 and/or 908 to read its medical device data including, for example, data representing the number of wash cycles that sling 904 has experienced. That data is then incremented to indicate another wash cycle has been performed (or is about to be performed) and the wash cycle data is written back to sling RFID tag 910. In alternative embodiments, scanners 912 and/or 908 can analyze the wash cycle data to determine if the sling 904 is near, at, and/or past its usable life based on comparing the wash cycle data to one or more predetermined thresholds.

Appropriate notifications and/or displays can be generated to indicate the lifetime status of the sling. These notifications/displays can take the form of one or more visual and/or auditory signals from washing machine 902 and/or scanner 908. The visual signals can include colored displays (e.g., yellow and/or red displays or lights (flashing or otherwise) indicating sling 904 requires attention, needs to be replaced soon, or replacement is required). The auditory signals can include, for example, one or more beeps, buzzes, voice notifications and/or alarms indicating the same. Other forms of notifications/displays can also be used. Still further, such notifications can also be stored as data on the RFID tag of the sling and/or transmitted to a remote server for device management (e.g., service, repair, inventory, re-ordering, etc.)

In this manner, injuries and unsafe conditions due to worn patient slings can be reduced or eliminated. Such slings can be identified through their RFID tag data and responsive actions can be taken to remove those slings from service, provide proper slings, and/or order new slings.

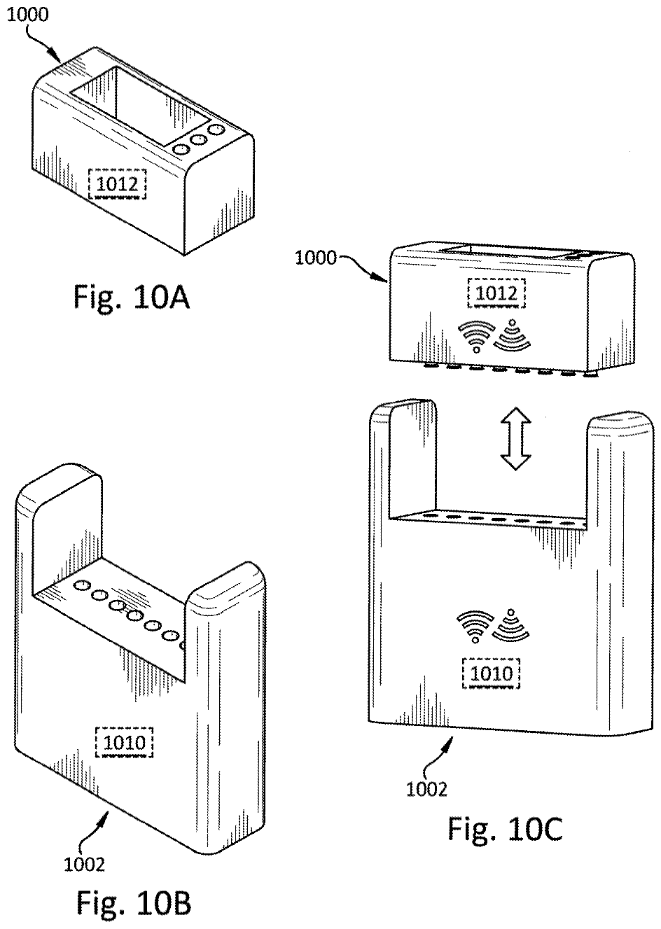

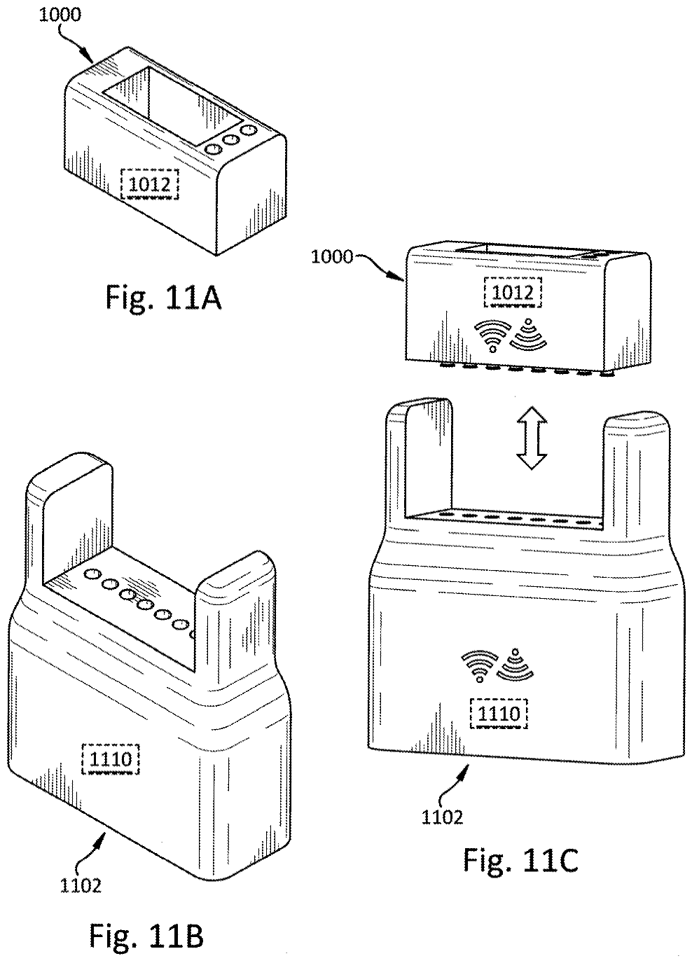

FIGS. 10A-10C and 11A-11C and 12 illustrate various embodiments of systems and methods for automatically configuring medical devices. FIGS. 10A-10C and 11A-11C illustrates embodiments of configuring a mix and match type medical device that may include a common head unit 1000 that is connectable to various base or accessory units/modules 1002 and/or 1102. In one example, the medical device can be an oxygen concentrator of the types disclosed in International Application No. PCT/US20/33591, which is hereby incorporated by reference. In one embodiment, head unit 1000 can include a controller and logic for operating one or more base or accessory units 1002 and/or 1102. Head unit 1000 includes a scanner 1012 similar to scanner 312 of FIG. 3 and/or other embodiments described herein. Various base units 1002 and/or 1102 include device data that can be stored on RFID tags 1010 and 1110. The device data stored in these RFID tags can include any of the previously described data including, for example, data sufficient to identify the base unit and/or one or more of the unit's operational parameters that would allow head unit 1000 to automatically configure itself to use the base unit to, for example, generate concentrated oxygen.

In the context of an oxygen concentrator, this data can include, for example, valve settings (e.g., open/close timing, etc.), flow settings (e.g., flow range, continuous, pulsed, high and low flow alarms, etc.), pressure settings (e.g., switch pressure, high and low pressure alarms, etc.), timing data, compressor speeds (variable, continuous, etc.) Because base units 1002 and 1102 can be designed to provide head unit 1000 with differing capabilities and capacities, head unit 1000 can automatically configure itself by scanning base unit RFID tags 1010 and/or 1110 and obtaining the necessary data to allow head unit 1000 to operate the base unit 1002 and/or 1102. For example, base unit 1002 may be arranged with components to provide a 3 liter per minute capacity oxygen concentrator. Base unit 1102 may be arranged with components to provide a 5 liter per minute capacity oxygen concentrator. The respective RFID tags of these base units can include data that includes one or more operational parameters to inform head unit 1000 how to configure itself to operate with the base unit.

In operation, head unit scanner 1012 scans for a responsive base unit and reads its medical device data, including operational parameters. If there are no alarm conditions/codes present in the base unit, head unit 1000 uses the operational data to automatically configure itself to work with the attached base unit. After configuration, head unit 1000 performs a start-up or warm-up sequence checking if the read operation parameters provide device operation within specific acceptable ranges associated with data in head unit 1000 controller and/or data read from the base unit RFID tag. If so, the head unit 1000 continues the start-up or warm-up sequence to completion and begins normal operation. As previously described, head unit 1000 can update or maintain RFID tag data associated with the base unit including, for example, storing updating usage, health and other data.

If head unit 1000 is unable to obtain device operation within acceptable ranges during the startup or warm-up sequence, an error is generated. In some embodiments, head unit 1000 may make several attempts using the read operational parameters of the base unit to obtain device operation within acceptable ranges before generating an error notification, message and/or data. The type of error may be written back to the base unit RFID tag for future reference.

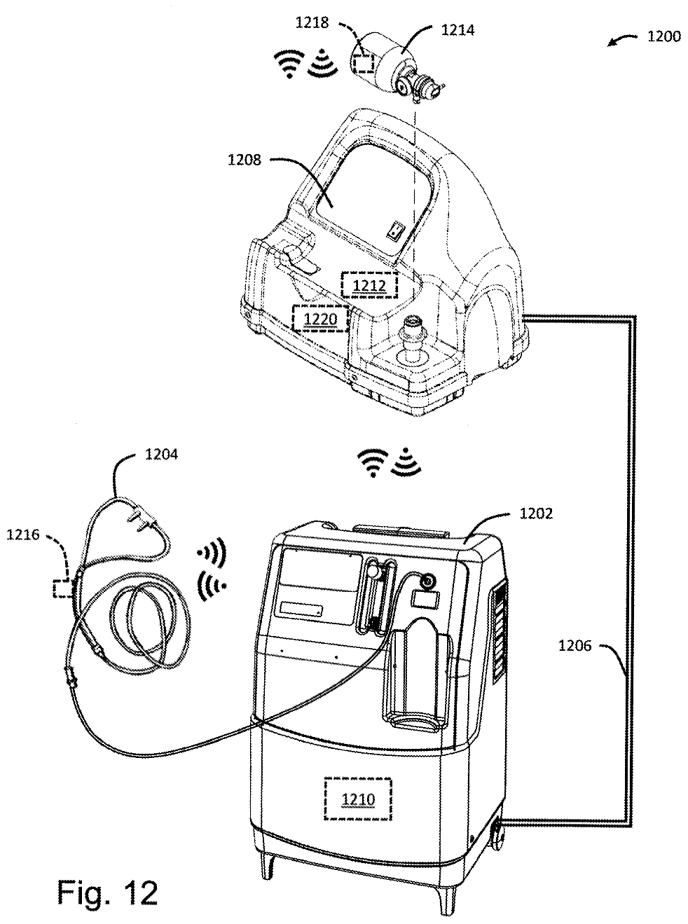

FIG. 12 illustrates another embodiment 1200 for automatically configuring medical devices. This embodiment relates to an oxygen concentrating system that can fill and refill oxygen bottles. Examples of such systems are disclosed in U.S. Pat. Nos. 5,988,165 and 6,302,107, which are incorporated herein by reference. This embodiment includes an oxygen concentrator 1202, a filling unit 1208, and a bottle or reservoir 1214. Optionally, this embodiment can include a breathing device such as nasal cannula 1204 (or nose/mouth oxygen mask). One or more of these components can include RFID tags (e.g., 1216, 1218, 1220) and one or more scanners 1210 for reading the RFID tags. In one embodiment, oxygen concentrator 1202 includes scanner 1210 that is any of the types previously described for reading and writing RFID tag data.

In operation, oxygen concentrator 1210 is connected via tubing 1206 to filling unit 1208. This provides filling unit 1208 with a source of concentrated oxygen. Filling unit 1208 includes an internal compressing device for taking the concentrated oxygen and further compressing it into oxygen storage bottle 1214. Oxygen storage bottle 1214 can be of various capacities or sizes and is used by patients that are “on the go” or ambulatory. These bottles are carried by the patient as the patient walks, moves, or travels from one location to another. A nasal cannula similar to 1204 (or other similar device) is connected to the bottle through a conserving device that provides the patient with concentrated oxygen while they are “on the go.”

In one embodiment, oxygen concentrator scanner 1210 can scan its surroundings to determine what types of components may be attached thereto. For example, scanner 1210 may detect the type of nasal cannula 1204 connected to the oxygen concentrator by reading the RFID tag 1216. RFID tag 1216 can include any of the previously described usage, health, and other data. For example, cannula RFID tag 1216 can include data that indicates the type of cannula including, for example, high flow, low flow, pediatric, adult, neonatal, etc. This data can be used by the controller of the oxygen concentrator to appropriately adjust the flow of oxygen to the patient. A variable position valve in oxygen concentrator 1202 can be used to lower the oxygen output flow rate for low flow, pediatric, and neonatal type cannula. Similarly, the variable position valve can be used to increase the flow rate for high flow and adult-type cannula. Furthermore, cannula usage data can be read, checked, and updated by scanner 1210 to ensure that the nasal cannula are not past their service life, nearing the end of their service life, or need to be replaced based on any of the previously described herein usage data exceeding predetermined thresholds. A display or other notification as previously described can be provided on oxygen concentrator 1202 to indicate the nasal cannula should be replaced or is nearing the time when it should be replaced.

Oxygen concentrator scanner 1210 can also scan its surroundings to determine the type of filling unit 1208 that is attached and/or the type bottle 1214 that is being used. In one embodiment, oxygen concentrator scanner 1210 can read and/or write data to RFID filling tag 1220 associated with filling unit 1208. Again, as previously described, filling unit RFID tag 1220 can include any of the previously described usage, health, and other data. The RFID tag data can be used to determine whether filling unit 1208 is an authorized component and/or whether it is acceptable for use based on its usage, health and other data. For example, the usage and/or health data can, as previously described herein, be compared to thresholds to determine if the filling unit is not past its service life, nearing the end of its service life, or needs to be replaced, repaired or serviced (due to end of life or error codes being present).

Oxygen concentrator 1202 can also automatically configure itself based on the presence/connection of filling unit 1208. For example, oxygen concentrator 1202 can configure itself to reduce its maximum patient output oxygen flow rate in order to provide filling unit 1208 with the appropriately high concentration of oxygen for storage in compressed bottle 1214. In one embodiment, this is accomplished by adjusting the position of a variable output valve to restrict the flow rate of oxygen gas being provided to the patient. In this manner, additional concentrated oxygen gas can be directed to filling unit 1208.

In another embodiment, oxygen concentrator 1202 can determine the runtime necessary to fill compressed storage bottle 1214 by reading the RFID tag 1218 to determine the size of the bottle (i.e., data identifying the size or capacity of the bottle). Once the RFID tag data has been read and the size of the bottle determined, oxygen concentrator 1202 can look up that information in its memory and obtain the time required to fill that size of bottle using filling unit 1208. Alternatively, oxygen concentrator 1202 can determine the filling time in real-time based on monitoring the actual flow rate of the concentrated oxygen gas being provided to filling unit 1208. The filling time can be displayed and/or updated on the display of oxygen concentrator 1202.

In yet another embodiment, filling unit 1208 can include a scanner 1212 of the types previously described herein. Scanner 1212 can read the RFID tags associated with the bottle 1214 and oxygen concentrator 1202 that is to be connected thereto. As previously described, scanner 1212 can use the read RFID tag data to determine whether concentrator 1202 is an authorized component and/or whether it is acceptable for use based on its usage, health and/or other data. For example, the usage and/or health data can, as previously described herein, be compared to thresholds to determine if the concentrator is not past its service life, nearing the end of its service life, or needs to be replaced, repaired or serviced (due to end of life or error codes being present). If the data indicates the oxygen concentrator is not fit for usage, filling unit 1208 can generate one or more notifications as previously described. Furthermore, filling unit 1208 may configure itself to not operate due to safety considerations if the oxygen concentrator is not fit for usage and to display such a notification.

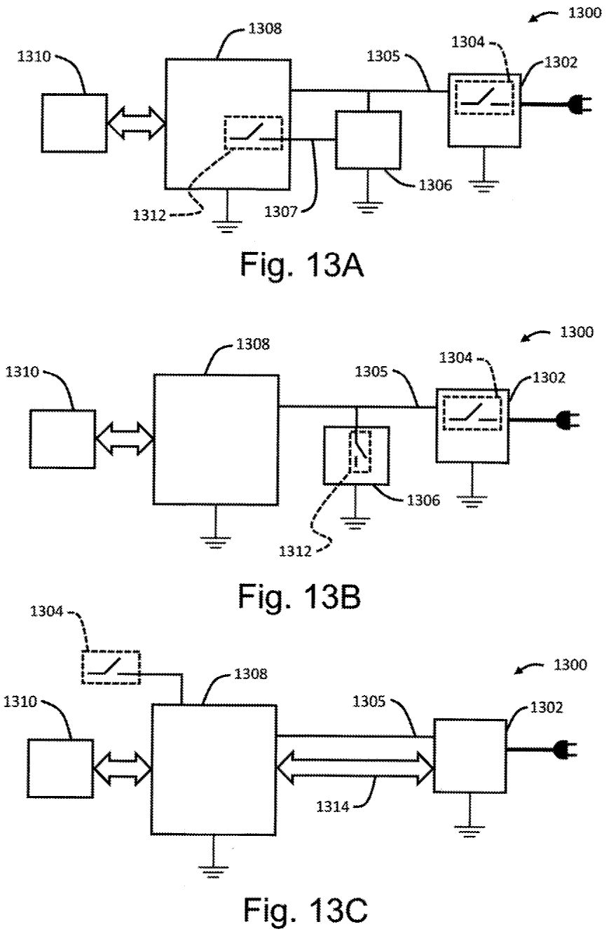

FIG. 13A illustrates one embodiment 1300 of a system and method for power management of RFID circuits. This embodiment provides power to the device controller so that RFID tag data can be read and/or written to after power to the device is turned off. The embodiment includes, for example, a power circuit 1302 for generating device power from a source such as, for example, a wall outlet, generator, or battery source. Power circuit 1302 includes a power or on/off switch 1304 that is used for turning on and off the device having the RFID tag. The embodiment also includes the controller circuitry 1308 for reading and/or writing data from/to RFID tag 1310. Controller circuitry 1308 normally receives its power from one or more electrical connections 1305 from power circuit 1302. A supplemental power circuit 1306 is also provided and arranged in parallel with power circuit 1306 and controller circuit 1308.

An auxiliary power switch 1312 is provided for when power from power circuit 1302 is interrupted or terminated. In one embodiment, switch 1312 connects controller circuitry 1308 to supplemental power circuit 1306 when controller circuit 1306 detects a drop or absence of power from power circuit 1302. In other embodiments, switch 1312 can be located within supplemental power circuit 1306 (FIG. 13B) and connects supplemental power circuit 1306 to controller circuitry 1308 when supplemental power circuit 1306 detects a drop or absence of power from power circuit 1302.

In operation, power circuit 1302 provides power to controller circuit 1308 for device operation and power to supplemental power circuit 1306. Supplemental power circuit 1306 includes an energy storage device such as a capacitor or inductor, which may be in series with a resistor. In alternate embodiments, supplemental power circuit 1306 can include a battery, which may be rechargeable by power circuit 1302. When switch 1304 is used to turn power off, power to controller circuit 1308 is interrupted or turned off thereby turning off the medical device. However, supplemental power circuit 1306 can still provide power to controller circuit 1308 for a predetermined time via electronic switch 1312 in order to allow controller circuit 1308 to read and/or write data (e.g., usage, health, identification, etc.) to RFID tag 1310. Switch 1312 can be any type of electronic switch including a power MOSFET or similar circuitry. In this manner, controller circuit 1308 can be configured to read and/or write data to the RFID tag when the power is turned off intentionally, unintentionally or when power is lost for other reasons (e.g., power failure or discontinuity) because power can be supplied by supplemental power circuit 1306.

FIG. 13C illustrates another embodiment where power switch 1304 is an input to controller circuit 1308. Controller circuit 1308 further communicates with power circuit 1302 via a data bus 1314. When power switch 1304 is actuated, controller circuit initiates its RFID tag data read and/write sequence to read/write device usage, health, and other data to RFID tag 1310. When the sequence is completed, controller circuit 1308 sends a message to power circuit 1302 that power can now be turned off. In any of these embodiments, power is maintained to controller circuit 1308 to allow RFID tag data to be read and/written to so that the RFID tag 1310 includes the last/latest set of data from the device.

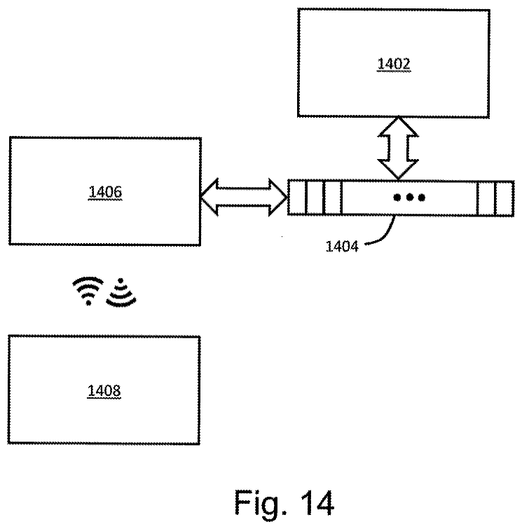

FIG. 14 illustrates one embodiment of a data tag structure or architecture for reading and/or writing data to an RFID tag. The data structure can be implemented in any form. In one embodiment, a data structure 1404 is provided having a length of 419 bytes, though more or less bits/bytes can be used. Data structure 1404 is used by device controller 1402 and the RFID tag 1406 to convey information between the two components. Similarly, RFID scanner device(s) 1408 uses data architecture 1404 to understand and convey information to and from the RFID tags. The data architecture can have any one or more of the assignments shown in Appendix A, which define, among other things, the device's usage, health, and other data. Also, the location of the data within the data structure can be changed or moved around. In this manner, the data structure provides an arrangement for conveying information between system components via RFID or other wireless technology.

While the present inventions have been illustrated by the description of embodiments thereof, and while the embodiments have been described in considerable detail, it is not the intention of the descriptions to restrict or in any way limit the scope of the inventions to such detail. Additional advantages and modifications will readily appear to those skilled in the art. Therefore, the inventions, in their broader aspects, are not limited to the specific details, the representative apparatus, and illustrative examples shown and described. Accordingly, departures can be made from such details without departing from the spirit or scope of the general inventive concepts.

APPENDIX A
RFID Bit Map (Note: RFID tag being used has 3328-bit user memory) 416 bytes

| Byte | | |
|---|---|---|
| number | Description | |
| 1 | Date Time | |
| 2 | Stamp from RTC | |
| 3 | | |
| 4 | | |
| 5 | | |
| 6 | | |
| 7 | | |
| 8 | | |
| 9 | Firmware | |
| 10 | Version ASCII | |
| 11 | | |
| 12 | | |
| 13 | | |
| 14 | | |
| 15 | Runtime | |
| 16 | Hours (uint) | |
| 17 | | |
| 18 | Per user | |
| 19 | runtime Hours | |
| 20 | SN (ASCII) | |
| 21 | or Integer | |
| 22 | | |
| 23 | | |
| 24 | | |
| 25 | | |
| 26 | | |
| 27 | | |
| 28 | | |
| 29 | | |
| 30 | Error Codes 1-8 | Currently Active Error Code/Error Code as Shutdown |
| 31 | Error Codes 9-16 | 1 bit for each error code (1 = active 0 = not active) |
| 32 | Error Codes 17-24 | |
| 33 | Error Codes 25-32 | |
| 34 | Error Codes 33-40 | |
| 35 | Error Codes 41-48 | |
| 36 | Error Codes 49-56 | |
| 37 | Error Codes 57-64 | |
| 38 | Error Codes 1-4 | Number of times each error code was triggered within |
| 39 | Error Codes 5-8 | the last week (each error 2 bits) |
| 40 | Error Codes 9-12 | 0 = 0 (error not triggered within the last week) |
| 41 | Error Codes 12-16 | 1 = 1-2 (error triggered 1-2 times) |
| 42 | Error Codes 17-20 | 2 = 3-5 (error triggered 3-5 times) |
| 43 | Error Codes 21-24 | 3 = 6 + (error triggered 6 or more times) |
| 44 | Error Codes 25-28 | |
| 45 | Error Codes 29-32 | |
| 46 | Error Codes 33-36 | |
| 47 | Error Codes 37-40 | |
| 48 | Error Codes 41-44 | |
| 49 | Error Codes 45-48 | |
| 50 | Error Codes 49-52 | |
| 51 | Error Codes 53-56 | |
| 52 | Error Codes 57-60 | |
| 53 | Error Codes 61-64 | |
| 54 | Error Codes 1-4 | Number of times each error code was triggered within |
| 55 | Error Codes 5-8 | the last Month (each error 2 bits) |
| 56 | Error Codes 9-12 | 0 = 0 (error not triggered within the last week) |
| 57 | Error Codes 12-16 | 1 = 1-3 (error triggered 1-2 times) |
| 58 | Error Codes 17-20 | 2 = 4-8 (error triggered 3-5 times) |
| 59 | Error Codes 21-24 | 3 = 9 + (error triggered 6 or more times) |
| 60 | Error Codes 25-28 | |
| 61 | Error Codes 29-32 | |
| 62 | Error Codes 33-36 | |
| 63 | Error Codes 37-40 | |
| 64 | Error Codes 41-44 | |
| 65 | Error Codes 45-48 | |
| 66 | Error Codes 49-52 | |
| 67 | Error Codes 53-56 | |
| 68 | Error Codes 57-60 | |
| 69 | Error Codes 61-64 | |
| 70 | Error Codes 1-4 | Number of times each error code was triggered since |
| 71 | Error Codes 5-8 | last per user runtime hours reset (each error 2 bits) |
| 72 | Error Codes 9-12 | 0 = 0 (error not triggered within the last week) |
| 73 | Error Codes 12-16 | 1 = 1-3 (error triggered 1-2 times) |
| 74 | Error Codes 17-20 | 2 = 4-8 (error triggered 3-5 times) |
| 75 | Error Codes 21-24 | 3 = 9 + (error triggered 6 or more times) |
| 76 | Error Codes 25-28 | |
| 77 | Error Codes 29-32 | |
| 78 | Error Codes 33-36 | |
| 79 | Error Codes 37-40 | |
| 80 | Error Codes 41-44 | |
| 81 | Error Codes 45-48 | |
| 82 | Error Codes 49-52 | |
| 83 | Error Codes 53-56 | |
| 84 | Error Codes 57-60 | |
| 85 | Error Codes 61-64 | |
| 86 | Flow Rate 0.5-1.5 | Hours used at each flow rate in the last week |
| 87 | Flow Rate 1.5-2.5 | (Int 1 byte each 0-256 hours) |
| 88 | Flow Rate 2.5-3.5 | |
| 89 | Flow Rate 3.5-4.5 | |
| 90 | Flow Rate 4.5+ | |
| 91 | Flow Rate 0.5-1.5 | Hours used at each flow rate in the last month |
| 92 | Flow Rate 1.5-2.5 | (Int 1 byte each 0-765 hours) |
| 93 | Flow Rate 2.5-3.5 | 1 bit = 3 hours |
| 94 | Flow Rate 3.5-4.5 | 0 = <1 hour |
| 95 | Flow Rate 4.5+ | 1 = 1-3 hours |
| | | 2 = 3-6 hours |
| | | ... |
| | | 255 = 762-765 hours |
| 96 | Flow Rate 0.5-1.5 | Hours used at each flow rate since last per user |
| 97 | | runtime hours reset (Int 2 byte2 each 0-65,535 hours) |
| 98 | Flow Rate 1.5-2.5 | |
| 99 | | |
| 100 | Flow Rate 2.5-3.5 | |
| 101 | | |
| 102 | Flow Rate 3.5-4.5 | |
| 103 | | |
| 104 | Flow Rate 4.5+ | |
| 105 | | |
| 106 | Average O2 | e.g. purity = 94.1% |
| 107 | purity last day | store as 941 |
| | 2 Bytes (int/10) | upon extraction divide by 10 to get 94.1% |
| 108 | Average O2 | e.g. purity = 94.1% |
| 109 | purity week day | store as 941 |
| | 2 Bytes (int/10) | upon extraction divide by 10 to get 94.1% |
| 110 | Average O2 purity | e.g. purity = 94.1% |
| 111 | Month day 2 Bytes | store as 941 |
| | (int/10) | upon extraction divide by 10 to get 94.1% |
| 112 | Average O2 purity since | e.g. purity = 94.1% |
| 113 | last user runtime hour | store as 941 |
| | reset day 2 Bytes (int/10) | upon extraction divide by 10 to get 94.1% |
| 114 | Average shift Pressure | Assume shift pressure = 28.2 PSI |
| | last day 0.5-1.5 LPM | Store as 28.2 * 5 = 141 |
| | | upon extraction divide by 5 to get shift pressure-> |
| | | 28.2 (+/− 0.1 PSI) |
| 115 | Average shift pressure | Assume shift pressure = 28.2 PSI |
| | last day 1.5-2.5 LPM | Store as 28.2 * 5 = 141 |
| | | upon extraction divide by 5 to get shift pressure-> |
| | | 28.2 (+/− 0.1 PSI) |
| 116 | Average shift pressure | Assume shift pressure = 28.2 PSI |
| | last day 2.5-3.5 LPM | Store as 28.2 * 5 = 141 |
| | | upon extraction divide by 5 to get shift pressure-> |
| | | 28.2 (+/− 0.1 PSI) |
| 117 | Average shift pressure | Assume shift pressure = 28.2 PSI |
| | last day 3.5-4.5 LPM | Store as 28.2 * 5 = 141 |
| | | upon extraction divide by 5 to get shift pressure-> |
| | | 28.2 (+/− 0.1 PSI) |
| 118 | Average shift pressure | Assume shift pressure = 28.2 PSI |
| | last day 4.5 + LPM | Store as 28.2 * 5 = 141 |
| | | upon extraction divide by 5 to get shift pressure-> |
| | | 28.2 (+/− 0.1 PSI) |
| 119 | Average shift Pressure | Assume shift pressure = 28.2 PSI |
| | last week 0.5-1.5 LPM | Store as 28.2 * 5 = 141 |
| | | upon extraction divide by 5 to get shift pressure-> |
| | | 28.2 (+/− 0.1 PSI) |
| 120 | Average shift pressure | Assume shift pressure = 28.2 PSI |
| | last week 1.5-2.5 LPM | Store as 28.2 * 5 = 141 |
| | | upon extraction divide by 5 to get shift pressure-> |
| | | 28.2 (+/− 0.1 PSI) |
| 121 | Average shift pressure | Assume shift pressure = 28.2 PSI |
| | last week 2.5-3.5 LPM | Store as 28.2 * 5 = 141 |
| | | upon extraction divide by 5 to get shift pressure-> |
| | | 28.2 (+/− 0.1 PSI) |
| 122 | Average shift pressure | Assume shift pressure = 28.2 PSI |
| | last week 3.5-4.5 LPM | Store as 28.2 * 5 = 141 |
| | | upon extraction divide by 5 to get shift pressure-> |
| | | 28.2 (+/− 0.1 PSI) |
| 123 | Average shift pressure | Assume shift pressure = 28.2 PSI |
| | last week 4.5 + LPM | Store as 28.2 * 5 = 141 |
| | | upon extraction divide by 5 to get shift pressure-> |
| | | 28.2 (+/− 0.1 PSI) |
| 124 | Average shift Pressure | Assume shift pressure = 28.2 PSI |
| | last month 0.5-1.5 LPM | Store as 28.2 * 5 = 141 |
| | | upon extraction divide by 5 to get shift pressure-> |
| | | 28.2 (+/− 0.1 PSI) |
| 125 | Average shift pressure | Assume shift pressure = 28.2 PSI |
| | last month 1.5-2.5 LPM | Store as 28.2 * 5 = 141 |
| | | upon extraction divide by 5 to get shift pressure-> |
| | | 28.2 (+/− 0.1 PSI) |
| 126 | Average shift pressure | Assume shift pressure = 28.2 PSI |
| | last month 2.5-3.5 LPM | Store as 28.2 * 5 = 141 |
| | | upon extraction divide by 5 to get shift pressure-> |
| | | 28.2 (+/− 0.1 PSI) |
| 127 | Average shift pressure | Assume shift pressure = 28.2 PSI |
| | last month 3.5-4.5 LPM | Store as 28.2 * 5 = 141 |
| | | upon extraction divide by 5 to get shift pressure-> |
| | | 28.2 (+/− 0.1 PSI) |
| 128 | Average shift pressure | Assume shift pressure = 28.2 PSI |
| | last month 4.5 + LPM | Store as 28.2 * 5 = 141 |
| | | upon extraction divide by 5 to get shift pressure-> |
| | | 28.2 (+/− 0.1 PSI) |
| 129 | Average shift Pressure | Assume shift pressure = 28.2 PSI |
| | since last user runtime | Store as 28.2 * 5 = 141 |
| | reset 0.5-1.5 LPM | upon extraction divide by 5 to get shift pressure-> |
| | | 28.2 (+/− 0.1 PSI) |
| 130 | Average shift pressure | Assume shift pressure = 28.2 PSI |
| | since last user runtime | Store as 28.2 * 5 = 141 |
| | reset 1.5-2.5 LPM | upon extraction divide by 5 to get shift pressure-> |
| | | 28.2 (+/− 0.1 PSI) |
| 131 | Average shift pressure | Assume shift pressure = 28.2 PSI |
| | since last user runtime | Store as 28.2 * 5 = 141 |
| | reset 2.5-3.5 LPM | upon extraction divide by 5 to get shift pressure-> |
| | | 28.2 (+/− 0.1 PSI) |
| 132 | Average shift pressure | Assume shift pressure = 28.2 PSI |
| | since last user runtime | Store as 28.2 * 5 = 141 |
| | reset 3.5-4.5 LPM | upon extraction divide by 5 to get shift pressure-> |
| | | 28.2 (+/− 0.1 PSI) |
| 133 | Average shift pressure | Assume shift pressure = 28.2 PSI |
| | since last user runtime | Store as 28.2 * 5 = 141 |
| | reset 4.5 + LPM | upon extraction divide by 5 to get shift pressure-> |
| | | 28.2 (+/− 0.1 PSI) |
| 134 | Max shift Pressure | Assume shift pressure = 28.2 PSI |
| | last day 0.5-1.5 LPM | Store as 28.2 * 5 = 141 |
| | | upon extraction divide by 5 to get shift pressure-> |
| | | 28.2 (+/− 0.1 PSI) |
| 135 | Max shift pressure | Assume shift pressure = 28.2 PSI |
| | last day 1.5-2.5 LPM | Store as 28.2 * 5 = 141 |
| | | upon extraction divide by 5 to get shift pressure-> |
| | | 28.2 (+/− 0.1 PSI) |
| 136 | Max shift pressure | Assume shift pressure = 28.2 PSI |
| | last day 2.5-3.5 LPM | Store as 28.2 * 5 = 141 |
| | | upon extraction divide by 5 to get shift pressure-> |
| | | 28.2 (+/− 0.1 PSI) |
| 137 | Max shift pressure | Assume shift pressure = 28.2 PSI |
| | last day 3.5-4.5 LPM | Store as 28.2 * 5 = 141 |
| | | upon extraction divide by 5 to get shift pressure-> |
| | | 28.2 (+/− 0.1 PSI) |
| 138 | Max shift pressure | Assume shift pressure = 28.2 PSI |
| | last day 4.5 + LPM | Store as 28.2 * 5 = 141 |
| | | upon extraction divide by 5 to get shift pressure-> |
| | | 28.2 (+/− 0.1 PSI) |
| 139 | Max shift Pressure | Assume shift pressure = 28.2 PSI |
| | last week 0.5-1.5 LPM | Store as 28.2 * 5 = 141 |
| | | upon extraction divide by 5 to get shift pressure-> |
| | | 28.2 (+/− 0.1 PSI) |
| 140 | Max shift pressure | Assume shift pressure = 28.2 PSI |
| | last week 1.5-2.5 LPM | Store as 28.2 * 5 = 141 |
| | | upon extraction divide by 5 to get shift pressure-> |
| | | 28.2 (+/− 0.1 PSI) |
| 141 | Max shift pressure | Assume shift pressure = 28.2 PSI |
| | last week 2.5-3.5 LPM | Store as 28.2 * 5 = 141 |
| | | upon extraction divide by 5 to get shift pressure-> |
| | | 28.2 (+/− 0.1 PSI) |
| 142 | Max shift pressure | Assume shift pressure = 28.2 PSI |
| | last week 3.5-4.5 LPM | Store as 28.2 * 5 = 141 |
| | | upon extraction divide by 5 to get shift pressure-> |
| | | 28.2 (+/− 0.1 PSI) |
| 143 | Max shift pressure | Assume shift pressure = 28.2 PSI |
| | last week 4.5 + LPM | Store as 28.2 * 5 = 141 |
| | | upon extraction divide by 5 to get shift pressure-> |
| | | 28.2 (+/− 0.1 PSI) |
| 144 | Max shift Pressure | Assume shift pressure = 28.2 PSI |
| | last month 0.5-1.5 LPM | Store as 28.2 * 5 = 141 |
| | | upon extraction divide by 5 to get shift pressure-> |
| | | 28.2 (+/− 0.1 PSI) |
| 145 | Max shift pressure | Assume shift pressure = 28.2 PSI |
| | last month 1.5-2.5 LPM | Store as 28.2 * 5 = 141 |
| | | upon extraction divide by 5 to get shift pressure-> |
| | | 28.2 (+/− 0.1 PSI) |
| 146 | Max shift pressure | Assume shift pressure = 28.2 PSI |
| | last month 2.5-3.5 LPM | Store as 28.2 * 5 = 141 |
| | | upon extraction divide by 5 to get shift pressure-> |
| | | 28.2 (+/− 0.1 PSI) |
| 147 | Max shift pressure | Assume shift pressure = 28.2 PSI |
| | last month 3.5-4.5 LPM | Store as 28.2 * 5 = 141 |
| | | upon extraction divide by 5 to get shift pressure-> |
| | | 28.2 (+/− 0.1 PSI) |
| 148 | Max shift pressure | Assume shift pressure = 28.2 PSI |
| | last month 4.5 + LPM | Store as 28.2 * 5 = 141 |
| | | upon extraction divide by 5 to get shift pressure-> |
| | | 28.2 (+/− 0.1 PSI) |
| 149 | Max shift Pressure since | Assume shift pressure = 28.2 PSI |
| | last user runtime reset | Store as 28.2 * 5 = 141 |
| | 0.5-1.5 LPM | upon extraction divide by 5 to get shift pressure-> |
| | | 28.2 (+/− 0.1 PSI) |
| 150 | Max shift pressure since | Assume shift pressure = 28.2 PSI |
| | last user runtime reset | Store as 28.2 * 5 = 141 |
| | 1.5-2.5 LPM | upon extraction divide by 5 to get shift pressure-> |
| | | 28.2 (+/− 0.1 PSI) |
| 151 | Max shift pressure since | Assume shift pressure = 28.2 PSI |
| | last user runtime reset | Store as 28.2 * 5 = 141 |
| | 2.5-3.5 LPM | upon extraction divide by 5 to get shift pressure-> |
| | | 28.2 (+/− 0.1 PSI) |
| 152 | Max shift pressure since | Assume shift pressure = 28.2 PSI |
| | last user runtime reset | Store as 28.2 * 5 = 141 |
| | 3.5-4.5 LPM | upon extraction divide by 5 to get shift pressure-> |
| | | 28.2 (+/− 0.1 PSI) |
| 153 | Max shift pressure since | Assume shift pressure = 28.2 PSI |
| | last user runtime reset | Store as 28.2 * 5 = 141 |
| | 4.5 + LPM | upon extraction divide by 5 to get shift pressure-> |
| | | 28.2 (+/− 0.1 PSI) |
| 154 | Min shift Pressure | Assume shift pressure = 28.2 PSI |
| | last day 0.5-1.5 LPM | Store as 28.2 * 5 = 141 |
| | | upon extraction divide by 5 to get shift pressure-> |
| | | 28.2 (+/− 0.1 PSI) |
| 155 | Min shift pressure | Assume shift pressure = 28.2 PSI |
| | last day 1.5-2.5 LPM | Store as 28.2 * 5 = 141 |
| | | upon extraction divide by 5 to get shift pressure-> |
| | | 28.2 (+/− 0.1 PSI) |
| 156 | Min shift pressure | Assume shift pressure = 28.2 PSI |
| | last day 2.5-3.5 LPM | Store as 28.2 * 5 = 141 |
| | | upon extraction divide by 5 to get shift pressure-> |
| | | 28.2 (+/− 0.1 PSI) |
| 157 | Min shift pressure | Assume shift pressure = 28.2 PSI |
| | last day 3.5-4.5 LPM | Store as 28.2 * 5 = 141 |
| | | upon extraction divide by 5 to get shift pressure-> |
| | | 28.2 (+/− 0.1 PSI) |
| 158 | Min shift pressure | Assume shift pressure = 28.2 PSI |
| | last day 4.5 + LPM | Store as 28.2 * 5 = 141 |
| | | upon extraction divide by 5 to get shift pressure-> |
| | | 28.2 (+/− 0.1 PSI) |
| 159 | Min shift Pressure | Assume shift pressure = 28.2 PSI |
| | last week 0.5-1.5 LPM | Store as 28.2 * 5 = 141 |
| | | upon extraction divide by 5 to get shift pressure-> |
| | | 28.2 (+/− 0.1 PSI) |
| 160 | Min shift pressure | Assume shift pressure = 28.2 PSI |
| | last week 1.5-2.5 LPM | Store as 28.2 * 5 = 141 |
| | | upon extraction divide by 5 to get shift pressure-> |
| | | 28.2 (+/− 0.1 PSI) |
| 161 | Min shift pressure | Assume shift pressure = 28.2 PSI |
| | last week 2.5-3.5 LPM | Store as 28.2 * 5 = 141 |
| | | upon extraction divide by 5 to get shift pressure-> |
| | | 28.2 (+/− 0.1 PSI) |
| 162 | Min shift pressure | Assume shift pressure = 28.2 PSI |
| | last week 3.5-4.5 LPM | Store as 28.2 * 5 = 141 |
| | | upon extraction divide by 5 to get shift pressure-> |
| | | 28.2 (+/− 0.1 PSI) |
| 163 | Min shift pressure | Assume shift pressure = 28.2 PSI |
| | last week 4.5 + LPM | Store as 28.2 * 5 = 141 |
| | | upon extraction divide by 5 to get shift pressure-> |
| | | 28.2 (+/− 0.1 PSI) |
| 164 | Min shift Pressure | Assume shift pressure = 28.2 PSI |
| | last month 0.5-1.5 LPM | Store as 28.2 * 5 = 141 |
| | | upon extraction divide by 5 to get shift pressure-> |
| | | 28.2 (+/− 0.1 PSI) |
| 165 | Min shift pressure | Assume shift pressure = 28.2 PSI |
| | last month 1.5-2.5 LPM | Store as 28.2 * 5 = 141 |
| | | upon extraction divide by 5 to get shift pressure-> |
| | | 28.2 (+/− 0.1 PSI) |
| 166 | Min shift pressure | Assume shift pressure = 28.2 PSI |
| | last month 2.5-3.5 LPM | Store as 28.2 * 5 = 141 |
| | | upon extraction divide by 5 to get shift pressure-> |
| | | 28.2 (+/− 0.1 PSI) |
| 167 | Min shift pressure | Assume shift pressure = 28.2 PSI |
| | last month 3.5-4.5 LPM | Store as 28.2 * 5 = 141 |
| | | upon extraction divide by 5 to get shift pressure-> |
| | | 28.2 (+/− 0.1 PSI) |
| 168 | Min shift pressure | Assume shift pressure = 28.2 PSI |
| | last month 4.5 + LPM | Store as 28.2 * 5 = 141 |
| | | upon extraction divide by 5 to get shift pressure-> |
| | | 28.2 (+/− 0.1 PSI) |
| 169 | Min shift Pressure since | Assume shift pressure = 28.2 PSI |
| | last user runtime reset | Store as 28.2 * 5 = 141 |
| | 0.5-1.5 LPM | upon extraction divide by 5 to get shift pressure-> |
| | | 28.2 (+/− 0.1 PSI) |
| 170 | Min shift pressure since | Assume shift pressure = 28.2 PSI |
| | last user runtime reset | Store as 28.2 * 5 = 141 |
| | 1.5-2.5 LPM | upon extraction divide by 5 to get shift pressure-> |
| | | 28.2 (+/− 0.1 PSI) |
| 171 | Min shift pressure since | Assume shift pressure = 28.2 PSI |
| | last user runtime reset | Store as 28.2 * 5 = 141 |
| | 2.5-3.5 LPM | upon extraction divide by 5 to get shift pressure-> |
| | | 28.2 (+/− 0.1 PSI) |
| 172 | Min shift pressure since | Assume shift pressure = 28.2 PSI |
| | last user runtime reset | Store as 28.2 * 5 = 141 |
| | 3.5-4.5 LPM | upon extraction divide by 5 to get shift pressure-> |
| | | 28.2 (+/− 0.1 PSI) |
| 173 | Min shift pressure since | Assume shift pressure = 28.2 PSI |
| | last user runtime reset | Store as 28.2 * 5 = 141 |
| | 4.5 + LPM | upon extraction divide by 5 to get shift pressure-> |
| | | 28.2 (+/− 0.1 PSI) |
| 174 | Altitude zone 1 | Hours used in each altitude zone in the last week |
| 175 | Altitude zone 2 | (Int 1 byte each 0-256 hours) |
| 176 | Altitude zone 3 | |
| 177 | Altitude zone 4 | |
| 178 | Altitude zone 5 | |
| 179 | Altitude zone 1 | Hours used at each flow rate in the last month |
| 180 | Altitude zone 2 | (Int 1 byte each 0-765 hours) |
| 181 | Altitude zone 3 | 1 bit = 3 hours |
| 182 | Altitude zone 4 | 0 = <1 hour |
| 183 | Altitude zone 5 | 1 = 1-3 hours |
| | | 2 = 3-6 hours |
| | | ... |
| | | 255 = 762-765 hours |
| 184 | Altitude zone 1 | |
| 185 | | |
| 186 | Altitude zone 2 | |
| 187 | | |
| 188 | Altitude zone 3 | Hours used at each flow rate since last per user |
| 189 | | runtime hours reset (Int 2 byte2 each 0-65,535 hours) |
| 190 | Altitude zone 4 | |
| 191 | | |
| 192 | Altitude zone 5 | |
| 193 | | |
| 194 | Average Temp last | e.g. Temperature = 94.1° F. |
| 195 | day 2 Bytes (int/10) | store as 941 |
| | | upon extraction divide by 10 to get 94.1° F. |
| 196 | Average Temp week | e.g. purity = 94.1% |
| 197 | day 2 Bytes (int/10) | store as 941 |
| | | upon extraction divide by 10 to get 94.1% |
| 198 | Average Temp Month | e.g. purity = 94.1% |
| 199 | day 2 Bytes (int/10) | store as 941 |
| | | upon extraction divide by 10 to get 94.1% |
| 200 | Average Temp since last | e.g. purity = 94.1% |
| 201 | user runtime hour reset | store as 941 |
| | day 2 Bytes (int/10) | upon extraction divide by 10 to get 94.1% |
| 202 | Max Temp last | e.g. Temperature = 94.1° F. |
| 203 | day 2 Bytes (int/10) | store as 941 |
| | | upon extraction divide by 10 to get 94.1° F. |
| 204 | Max Temp week | e.g. purity = 94.1% |
| 205 | day 2 Bytes (int/10) | store as 941 |
| | | upon extraction divide by 10 to get 94.1% |
| 206 | Max Temp Month | e.g. purity = 94.1% |
| 207 | day 2 Bytes (int/10) | store as 941 |
| | | upon extraction divide by 10 to get 94.1% |
| 208 | Max Temp since last | e.g. purity = 94.1% |
| 209 | user runtime hour reset | store as 941 |
| | day 2 Bytes (int/10) | upon extraction divide by 10 to get 94.1% |
| 210 | Min Temp last | e.g. Temperature = 94.1° F. |
| 211 | day 2 Bytes (int/10) | store as 941 |
| | | upon extraction divide by 10 to get 94.1° F. |
| 212 | Min Temp week | e.g. purity = 94.1% |
| 213 | day 2 Bytes (int/10) | store as 941 |
| | | upon extraction divide by 10 to get 94.1% |
| 214 | Min Temp Month | e.g. purity = 94.1% |
| 215 | day 2 Bytes (int/10) | store as 941 |
| | | upon extraction divide by 10 to get 94.1% |
| 216 | Min Temp since last user | e.g. purity = 94.1% |
| 217 | runtime hour reset day | store as 941 |
| | 2 Bytes (int/10) | upon extraction divide by 10 to get 94.1% |
| 218 | Sieve Bed | int |
| | Health (0-100%) | |
| 219 | Maintenance timer 1 | Timer to indicate service needed |
| | | (filter replacement, compressor service, ...) |
| 220 | Maintenance timer 2 | |
| | | (filter replacement, compressor service, ...) |
| 221 | Maintenance timer 3 | Timer to indicate service needed |
| | | (filter replacement, compressor service, ...) |
| 222 | Maintenance timer 4 | Timer to indicate service needed |
| | | (filter replacement, compressor service, ...) |
| 223 | Maintenance timer 5 | Timer to indicate service needed |
| | | (filter replacement, compressor service, ...) |
| 224 | Maintenance timer 6 | Timer to indicate service needed |
| | | (filter replacement, compressor service, ...) |
| 225 | Maintenance timer 7 | Timer to indicate service needed |
| | | (filter replacement, compressor service, ...) |
| 226 | Maintenance timer 8 | Timer to indicate service needed |
| | | (filter replacement, compressor service, ...) |
| 227 | Maintenance timer 9 | Timer to indicate service needed |
| | | (filter replacement, compressor service, ...) |
| 228 | Maintenance timer 10 | Timer to indicate service needed |
| | | (filter replacement, compressor service, ...) |
| 229 | Maintenance timer 11 | Timer to indicate service needed |
| | | (filter replacement, compressor service, ...) |
| 230 | Maintenance timer 12 | Timer to indicate service needed |
| | | (filter replacement, compressor service, ...) |
| 231 | Reserved for Future use | |
| ... | | |
| 315 | | |
| 316 | Reserved for writing | |
| ... | data to the unit from | |
| 416 | the RFID Reader. This | |
| | could include updating | |
| | the unit serial number | |
| | for service. | |

What is claimed:

1. A gas concentrating system comprising:
a gas separation assembly;
a controller;
a data storage in communication with the controller and wirelessly accessible by external devices; and
logic for storing system usage data in the data storage.

2. The system of claim 1, wherein the system usage data comprises compressor usage time data.

3. The system of claim 1, wherein the system usage data comprises alarm data.

4. The system of claim 1, wherein the system usage data comprises oxygen data.

5. The system of claim 1, wherein the system usage data comprises shift pressure data.

6. The system of claim 1, wherein the system usage data comprises temperature data.

7. The system of claim 1, wherein the system usage data comprises location data.

8. The system of claim 1, wherein the system usage data comprises patient data.

9. The system of claim 1, further comprising logic for allowing an external device to wirelessly access and store data to the data storage.

10. The system of claim 1, wherein the data storage is a radio frequency identification (RFID) device.

11. A system for managing medical devices comprising:
a controller;
a memory in communication with the controller;
a scanner for wirelessly communicating with one or more medical devices;
logic for reading medical device data; and
logic for determining if the medical device needs to be serviced based on the read data.

12. The system of claim 11, wherein the logic for determining if the medical device needs to be serviced based on the read data comprises logic for determining if at least one alarm or error code is present in the read data.

13. The system of claim 11, wherein the logic for determining if the medical device needs to be serviced based on the read data comprises logic for determining if the read data exceeds a medical device usage threshold.

14. The system of claim 11, wherein the logic for determining if the medical device needs to be serviced based on the read data comprises logic for determining if the read data indicates a compressor usage time is exceeds a threshold.

15. The system of claim 11, wherein the logic for determining if the medical device needs to be serviced based on the read data comprises logic for determining the read data indicates an oxygen content value is below a threshold.

16. The system of claim 11, wherein the logic for determining if the medical device needs to be serviced based on the read data comprises logic for determining if the read data indicates a shift pressure value is outside of a threshold range.

17. A method of managing at least one medical device, comprising:
wirelessly reading usage data from a medical device;
comparing the usage data to one or more thresholds; and
based on the comparison, identifying if the device can be placed back in inventory or the device is need of service.

18. The method of claim 17, wherein comparing the usage data to one or more thresholds comprises comparing compressor usage time data to a time threshold.

19. The method of claim 17, wherein comparing the usage data to one or more thresholds comprises comparing oxygen data to an oxygen threshold.

20. The method of claim 17, wherein comparing the usage data to one or more thresholds comprises comparing shift pressure data to an pressure range threshold.
